(19) United States
(12) Patent Application Publication
(10) Pub. No.: US 2012/0183620 A1
(43) Pub. Date: Jul. 19, 2012

(54) MESOPOROUS DRUG DELIVERY SYSTEM USING AN ELECTRICALLY CONDUCTIVE POLYMER

(75) Inventors: Youngnam Cho, Koyang (KR); Riyi Shi, West Lafayette, IN (US); Albena Ivanisevic, West Lafayette, IN (US); Richard Borgens, Delphi, IN (US)

(73) Assignee: Purdue Research Foundation, West Lafayette, IN (US)

(21) Appl. No.: 13/381,201

(22) PCT Filed: Jun. 30, 2010
(86) PCT No.: PCT/US10/40691

§ 371 Date: Mar. 23, 2012

Related U.S. Application Data
(60) Provisional application No. 61/221,756, filed on Jun. 30, 2009.

(51) Int. Cl. A61K 9/14; C25D 1/08; A61K 38/18; A61P 25/00; B82Y 5/00

(52) U.S. Cl. 424/490; 514/8.4; 205/75; 977/788; 977/906

(57) ABSTRACT

The present application relates to Nanoparticle bioengineering techniques were used to produce a non-toxic polypyrrole composition having two-dimensional and three-dimensional structures that can optionally be co-polymerized with carboxylic acid moieties to possess hydrophilicity. Likewise, such polypyrrole/carboxylic acid structures may be further modified with neural growth factors to create treatment surfaces that can promote growth an differentiation of cells such as neurons.

### PRIORITY

The present application claims priority to U.S. Provisional Patent Application Ser. No. 61/221,756, Titled A Mesoporous Silica Nanosphere-Based Drug Delivery System Using An Electrically Conduction Polymer and filed on Jun. 20, 2009, the contents of which are hereby incorporated by reference.

### BACKGROUND

### Introduction

Electrically conducting polymers have attracted considerable attention for numerous applications as biomedical tools, biosensors, batteries, and microelectronic devices. One such compound, polypyrrole (Ppy), has become prominent in biology and medicine as a potential electrically conductive polymer due to its distinctive non-toxic properties and biocompatibility. The native conductivity of Ppy can be altered from an inherent insulating feature (σ_{dc}≦1×10⁻⁷ S cm⁻¹) to the level of metallic conductance (σ_{dc}≦1×10² S cm⁻¹) during oxidative polymerization which readily involves the entrapment of a variety of anions and cations as a dopant. These facts are important to consider since electrical current is used in modern treatments of central nervous system (CNS). Useful biomolecules such as growth factors and/or anti-inflammatory drugs could theoretically be delivered to damaged tissues by the same electrodes used to direct the electrical therapy. Considerable improvements have been achieved by combining drug delivery systems with electrical stimulation through a Ppy backbone, where biomolecules are incorporated, or doped, into Ppy films. These can subsequently be released as a result of the reduction of the Ppy.

However, there are many issues to resolve in order to fully realize the promises of Ppy delivery systems, including: (i.) low encapsulation and rapid release of a drug associated with the limited surface area, (ii.) the inherent hydrophobic nature of conductive polymers, and iii. the lack of plentiful functional groups for significant surface modification and utilization. To enhance the affinity of biomolecules for Ppy surfaces, direct or indirect surface functionalization with bioactive peptides, proteins, or enzymes is one barrier that must be overcome. Despite the advances in surface modification techniques, this step is complex and time-consuming and remains challenging. In addition, electrochemical deposition using a solution composed of a biological entity and pyrrole monomers restrict homogeneous drug distribution throughout the polypyrrole surface and interfere with electrochemical control of various parameters such as film thickness, loading concentration, and diffusion of the biomolecules of interest. To date, porous Ppy structures have been fabricated through electrochemical deposition of pyrrole in the presence of polymethyl methacrylate, tetraethyylammonium percholate (TEAP), or poly (acrylic acid) and utilized as a biomaterial.

### BRIEF DESCRIPTION OF THE DRAWINGS

FIG. 1(a) displays a schematic illustration for the preparation of carboxylic acid-terminated polypyrrole coating in the presence of MSN/NGF through electrochemical deposition. The electropolymerization was conducted in an aqueous solution of a mixture of 0.1 M pyrrole (Py) monomer and 0.1 M pyrrole-α-carboxylic acid (Py-α-COOH) by applying a steady potential at 0.7 V and changing it to 2.0 V in the subsequent reduction profile to induce a coupling reaction between Py and Py-α-COOH.

FIG. 1(b) displays TEM images of MSNs incorporated with NGF. The inset shows the as-synthesized MSNs.

FIG. 1(c) displays an SEM image of particle arrays obtained by a convective evaporation where capillary forces are the most dominant factor for confining particles in layered arrays.

FIG. 1(d) SEM image of the electropolymerization of carboxylic acid-functionalized polypyrrole in the presence of NGF-loaded MSNs as a template, which is designated as a Ppy/COOH-MSN/NGF.

FIG. 2(a) displays a fluorescence image of a Ppy/COOH-MSN/NGF-FITC surface.

FIG. 2(b) The cumulative NGF release behavior over a week from Ppy/COOH-MSN/NGF composite in the presence or absence of electrical stimulation.

FIG. 3 displays a high-resolution XPS for surface deconvolution analysis of (a) and (b) C 1s and (c) and (d) N 1s spectra of a Ppy/COOH (a and c) and a Ppy/COOH-MSN/NGF (b and d) film.

FIG. 4(a) displays a typical SEM image of the resulting macroporous Ppy/COOH film using a template composed of MSNs with a mean diameter of 150 nm. FIG. 4(b) Cyclic voltammogram of 5 mM Fe(CN)₆^{3-/4-} at bare ITO (red), a Ppy/COOH-MSN composite film (brown), a porous Ppy/COOH film (green), and a porous Ppy/COOH-NGF film (blue) at a scan rate of 50 mV sec⁻¹. High-resolution XPS of (c) and (d) C 1s and (e) and (f) N 1s spectra of a porous Ppy/COOH (c and e) and a porous Ppy/COOH coupled with NGF (d and f).

FIG. 5 displays optical micrographs of PC 12 cell cultured on (a) and (b) porous Ppy/COOH films surface conjugated with NGF and (c) and (d) Ppy/COOH-MSN/NGF films in the absence (a and c) or presence of electrical stimulation (b and d).

FIG. 6(a) displays a graphical representation of the percentage of cells with neurites that extend from PC 12 cells grown on various types of Ppy/COOH films in the absence or presence of electrical stimulation after 7 days in culture.

FIG. 6(b) displays a graphical representation of the percentage of cells neurite length of PC 12 cells grown on various types of Ppy/COOH films in the absence or presence of electrical stimulation after 7 days in culture.

### DETAILED DESCRIPTION

According to at least one embodiment, the deposition of carboxylic acid-terminated conducting polymers in two or three dimensional structures made up of colloidal particles is performed, resulting in a porous polypyrrole surface. By way of nonlimiting example, porous polypyrrole surface is produced, first through the production of ordered arrays of mesoporous silica nanoparticles (“MSNs”) as a template. According to at least one embodiment, thereafter polypyrrole (Ppy) and/or carboxylic acid-terminated polypyrrole (Ppy/COOH) is electrochemically prepared on the MSN template.

According to at least one embodiment, the electropolymerization of a polypyrrole (Ppy) and/or carboxylic acid-terminated polypyrrole (Ppy/COOH) is performed in an aqueous solution of a mixture of a dopant ion, which could optionally be neural growth factors (“NGFs”), a combination of NGFs and MSNs, and/or a combination of PPY/COOH-MSN/NGF. According to at least one embodiment, the ordered arrays of MSNs are optionally selected to remain within the resultant Ppy film or structure, or are optionally treated to remove the MSN template, resulting in a porous conductive two or three dimensional structure. According to at least one embodiment, the resultant Ppy/COOH-MSN/NGF structure is treated through dipping the structure in an acidic treatment to remove the NGF template.

### Example 1

### Fabrication of MSNs

According to at least one embodiment, a mesoporous silica nanopartical according to the present application was produced. All chemicals were purchased from Sigma-Aldrich unless otherwise specified. MCM 41-type mesoporous silica nanoparticles (MSNs) were synthesized according to those procedures set forth in Slowing I, Trewyn B and Lin V 2007 J. Am. Chem. Soc. 129 8845-9. An aqueous solution containing cetyltrimethylammonium bromide (CTAB) and ammonia was stirred at 80° C. for 2 hrs, and then tetraethyl orthosilicate (TEOS) was slowly added to the mixture. Subsequently, mesitylene was added as a pore-expanding agent, and the solutions were stirred at elevated temperature for another 3 hr. The resulting white precipitate collected by repetitive filtration was rinsed with water and dried at 100° C. for 12 hr. Finally, an acidic extraction method (0.75 mL concentrated HCl/100 mL methanol solution) was performed overnight to remove the CTAB template. For NGF immobilization, 5 mL of PBS solutions with 100 μL of NGF 2.5 S (Invitrogen, 100 μg/mL) was stirred for 6 hrs at room temperature in the presence of 20 mg of dried MSN. The suspensions were centrifuged and dried overnight at room temperature under vacuum. Transmission electron microscopy (TEM) confirmed that freshly prepared colloids showed a mean diameter of 150 nm with regularity in shape and size.

### Example 2

### Preparation of Various Types of Carboxylic Acid-Terminated Polypyrrole (Ppy/COOH) Films in the Presence of Templates Comprising MSN/NGFs

According to at least one exemplary embodiment, three dimensional particle arrays were obtained by a self-assembly technique where capillary forces are the most dominant factor for confining particles in layered arrays. A clean ITO surface (Indium tin oxide, available from Delta Technologies) was dipped in a 15 ml of MSNs, MSN/NGF, or MSN/NGF-FITC re-dispersed in ethanol with a density of 5 mg/ml, where FITC-labeled NGF was prepared according to the procedure by Gomez N and Schmidt C E 2006 Nerve growth factor-immobilized polypyrrole: Bioactive electrically conducting polymer for enhanced neurite extension J Biomed Mater Res A. 81A 135-49. The template with a uniform deposition of silica nanoparticles was achieved at room temperature over ˜5 days by slow ethanol evaporation. The film thickness was adjustable due to precise control of the particle size and the total number of layers. Finally, the template was air-dried for at least 1 day and kept in a vacuum desiccator until use. Polypyrrole (Ppy) was electrochemically prepared on a silica nanoparticle assembled ITO surface using 604 model potentiostat (CH Instruments). A template, platinum gauze, and saturated calomel electrode were employed as a working, a counter, and a reference electrode, respectively. The electropolymerization of carboxylic acid-terminated polypyrrole (Ppy/COOH) was conducted on top of MSN/NGF assembled ITO surfaces in an aqueous solution of a mixture of 0.1 M pyrrole (Py) monomer, 0.1 M pyrrole-α-carboxylic acid (Py-α-COOH), and 0.1 M sodium salt of poly (styrene sulfonate) (PSS) as a dopant ion, which was designated as a Ppy/COOH-MSN/NGF. This was initially achieved by applying a steady potential at 0.7 V and changing it to 2.0 V in the subsequent reduction profile to induce a coupling reaction between Py and Py-α-COOH. These films were immediately rinsed with deionized water and dried under nitrogen to avoid any further deposition.

According to at least one embodiment, NGF is into the pores of MSNs prior to deposition of the MSN template. MSNs synthesized with large-pore diameters have attracted much attention as an inorganic host material to encapsulate large biomolecules, such as enzymes, proteins, and even cells. The adsorption of substances by an inorganic matrix improves their stability by protecting them from the systematic circulation and consequently increases the therapeutic effect. MSNs possess some inherent advantages such as their nontoxic and biocompatible nature, adjustable pore size, large surface-to-volume ratio, and chemical stability with tunable degradation rates. The encapsulation of NGF (MW 13000) into the well-ordered internal structure of an MSN was performed by favorable electrostatic interaction between free silanol groups on the wall of pore and positively charged amine groups of NGF at pH 7.0. The confinement of NGF to the MSN matrix was confirmed by TEM, N2 adsorption, and XPS. As-synthesized CTAB removed MSNs exhibit well-ordered pore structure with uniform mesopores whereas NGF-loaded MSNs demonstrated filling, indicated by the presence of NGF inside the pore channels. The incorporation of NGF is further highlighted by the comparison of the physical properties of as-synthesized MSN and MSN-NGF using a N2 adsorption/desorption isotherm. The BET test revealed 1040 m2 g-1 surface area and 1.93 cm³ g-1 of total pore volume of as-synthesized MSN with 4.71 nm of pore diameter. This indicated that MSNs possess enough space for drug molecules. The marked uptake of NGF lowered the surface area and total pore volume by approximately 60% and 80% respectively. Moreover, the reduction in average pore diameter strongly suggests that the majority of the pore walls were covered with NGF molecules. The particle size of MSN in PBS was measured by dynamic light scattering. Two different diameter distributions were observed: 101 and 125 nm corresponding to before and after NGF uptake respectively with approximately 31.5% of NGF encapsulation efficiency.

For the preparation of porous Ppy/COOH films, the Ppy/COOH-MSN template was dipped in the acidic treatment with 20% HF for 24 hr. The resulting porous conductive film with carboxylic acid derivatives was fabricated and air-dried. The NGF conjugated porous Ppy/COOH-NGF films were achieved through a two-step modification process. In the first step, carboxylic acid-terminated porous Ppy/COOH surfaces were incubated in a 50 mM MES buffer solutions containing 50 mg/ml N-hydroxysuccinimide (NHS) aqueous solution and 0.3 M1-ethyl-3-(3-dimethyl-aminopropyl) carbodiimidi hydrochloride (EDC) for 6 hrs at room temperature. Subsequently, estered Ppy/COOH was re-incubated in a 50 mM MES buffer and 100 μg/mL of NGF 2.5 S (Invitrogen) at room temperature for 3 hrs. The NGF-coated surface was washed with deionized water and dried under nitrogen. The concentration of NGF functionalized on a porous Ppy/COOH surface was determined by measuring the intensity of NGF-FITC with a fluorescence microscope using excitation/barrier wavelengths of 490/520 nm and by interpolating with the standard plot. The standard curve in terms of surface concentration (ng/mm²) was prepared by measuring the fluorescence (minus background) intensity of experimental Ppy surfaces that are prepared by covalently coupling with known amounts of NGF-FITC and was statistically quantified by capturing the FITC labeling using NIH Image™ software.

### Example 3

### The Release Profile of NGF from Ppy/COOH-MSN/NGF Composite, and Efficacy in Triggering Cell Proliferation and Neurite Extension

According to one exemplary embodiment, the release behavior of NGF from Ppy/MSN-MSN/NGF composite was evaluated using a commercially available sandwich ELISA kit (Millipore). The PBS solution containing Ppy/MSN-NGF was kept at 37° C. until an aliquot was taken from the suspension at different times. The aliquots collected were evaluated with ELISA immunoassay in undiluted aqueous samples. The intracellular NGF content was calculated based on the absorbance at 450 nm. Each NGF “release” experiment was performed in triplicate.

According to at least one exemplary embodiment, the efficacy in triggering cell proliferation of a resultant Ppy/MSN-MSN/NGF composite or Ppy/COOH-NGF films was tested. PC 12 cells (density of 1×10⁶ cells/mL) were grown in Dulbecco's modified eagle's medium (DMEM; Invitrogen) supplemented with 12.5% horse serum, 2.5% fetal bovine serum, 50 U/ml penicillin, and 5 mg/ml streptomycin at an incubator setting of 5% CO₂ and 37° C. After trypsinization and centrifugation, cell pellets were resuspended in tissue culture dishes containing Ppy films to observe both cell proliferation and neurite extension as a function of time. During these experiments, PC 12 cells grown on bare substrates were supplied with an appropriate amount of NGF solution (50 ng/mL) to the culture medium whereas cells cultured on Ppy/COOH-MSN/NGF and porous Ppy/COOH-NGF films were maintained without the addition of NGF. Finally, cells were observed and photographed using phase-contrast microscopy.

### Example 4

### Testing of Electrical Stimulation

PC 12 cells with a density of 1×10⁴ cells/cm² were grown on various types of Ppy films and incubated for 24 hr to permit cell adhesion and neurite extension as a result of electrical stimulation of the films. Ppy substrates were placed in borosilicate coverglass chambers containing three electrodes; a reference electrode (Ag/AgCl), a counter electrode (Pt), and a working electrode (Ppy film). A constant voltage of 0.1 V for 6 hr was applied to the Ppy films to induce a burst outgrowth effect from the composite. Neurite length was analyzed after 1 day of stimulation. All experiments were performed in triplicate.

According to at least one embodiment, MCM-41 type mesoporous silica nanoparticles (MSNs) provided extremely high surface areas (>1000 m²/g) and tunable pore diameter in the range of 2˜10 nm. Furthermore, a large surface-to-volume ratio allowed for effective entrapment of biological compounds into the pores of MSNs while retaining their bioactivity. Applicants achieved the synthesis of MCM-41-type MSN materials with large pore diameters (4.3 nm) and subsequent encapsulation of NGF inside the channels by favorable electrostatic interactions.

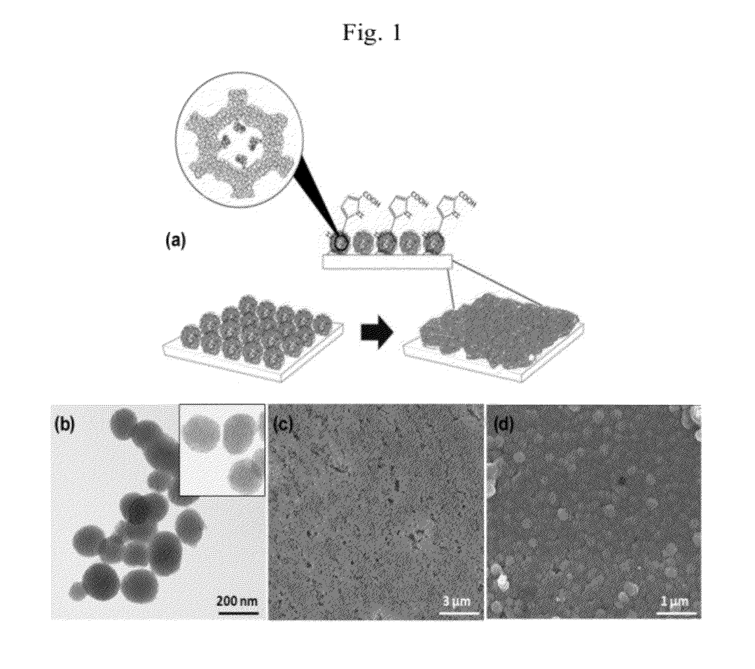

As shown in FIG. 1(b), NGF-loaded MSNs with a broad range of diameters revealed complete filling when compared to that of as-synthesized MSNs, thus emphasizing the confinement of NGF of the mesopores. Additionally, applicants used two complementary techniques, N₂ adsorption and XPS, to verify the adsorption of NGF to the MSN matrix. Subsequently, templates composed of NGF-loaded MSNs on ITO surfaces were constructed taking advantage of a convective self-assembly technique. Typical SEM images showed close-packed arrangements of MSNs, demonstrating that silica particles driven by capillary forces were able to serve as templates for the formation of conductive polymers (FIG. 1 c). The variation of evaporation pressure in the dispersion solution was used to determine the surface quality of a colloidal template. For instance, rapid evaporation of ethanol containing silica particles resulted in poor uniformity of the colloidal arrangement with low surface coverage. In contrast, slow ethanol evaporation favored better three-dimensional organization into large ordered domains along with stable interparticle forces. Because the use of conductive polymers in biomedical research is of great interest, the creation of effective surface areas with positive features of well-defined architectures and high surface area is critical in determining their electrical and physicochemical properties to promote better integration with cells. To this end, particle arrays were further used as a template to achieve carboxylic acid-terminated conductive polypyrrole (Ppy) surfaces through electrochemical co-deposition using pyrrole monomers (Pys) and carboxylic acid-terminated pyrrole monomers (Py-COOHs), as illustrated in FIG. 1(a).

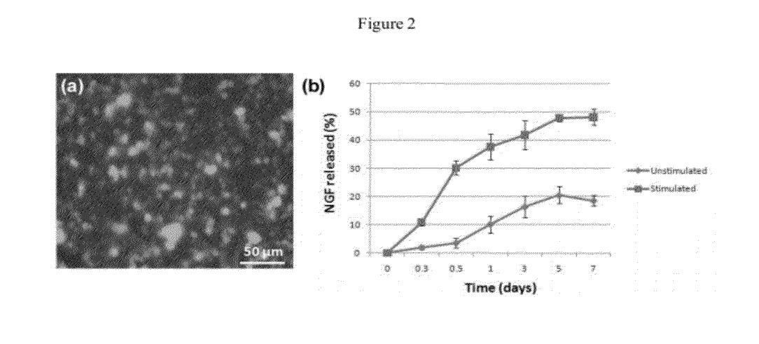

In consideration of the oxidative potentials of Py (+0.8 V) and Py-COOH (+1.38 V), the increase in potential magnitude from +0.7 V to +2.0 V permitted the co-electropolymerization process of Py and Py-COOH in subsequent potential scans as shown in FIG. 1(d). Such monomers could be infiltrated into the colloidal templates, especially the interstitial channels of a template, to obtain uniform conductive films during oxidative polymerization. Though electrochemical deposition is an effective method for controlling film growth, governing the infiltration of the precursor polymer is not simple because physical properties (including viscosity and concentration of precursors) often cause incomplete filling. The resulting Ppy/COOH films revealed spatially homogeneous dispersion of NGF on the surfaces by fluorescence microscopy (FIG. 2a).

A uniform fluorescent molecule distribution within a Ppy surface has been attributed to the appearance of MSNs incorporated with FITC-labeled NGF. We also assessed the effect of electrical stimulation on the release of NGF from Ppy composites over one week since the release of chemical substances (e.g., drugs, nerve growth factors, neurotrophic factors, etc) embedded in conducting polymer films can be manipulated in response to electrical potential by inducing a reversible expansion/contraction in conjugated polymers. Our study showed that regardless of electrical stimulation, a gradual increase in NGF release was observed for the next 7 days. However, it should be highlighted that the stimulated surface showed a significant improvement in the release profile resulting from the redox characteristics of the polypyrrole. Next, the chemical composition of carboxylic-acid functionalized Ppy surfaces was analyzed by XPS. Initially we examined: carboxylic acid-terminated Ppy (Ppy/COOH) and carboxylic acid-terminated Ppy incorporated with MSN-NGF (Ppy/COOH-MSN/NGF). In order to understand the composition of the surface, high-resolution spectra were recorded for the main core-level peaks of C 1s and N 1s because they enabled us to evaluate the chemical structure of the species present on the surface.

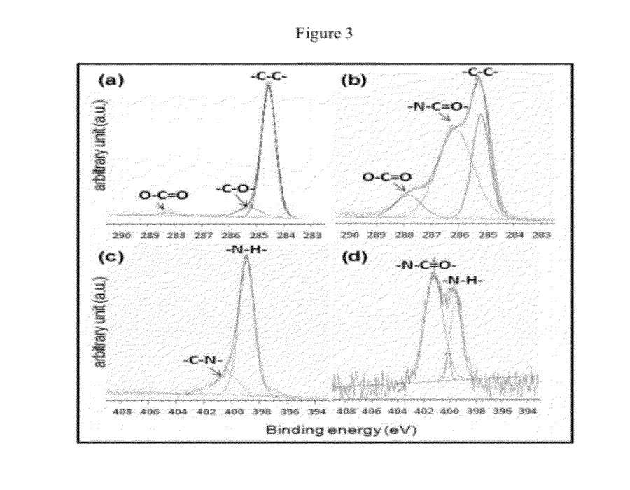

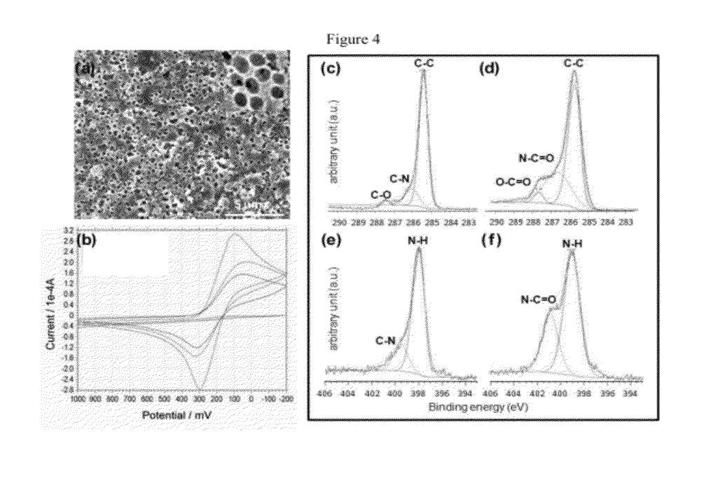

Turning now to FIG. 3, a high-resolution XPS for surface deconvolution analysis of (a) and (b) C 1s and (c) and (d) N 1s spectra of a Ppy/COOH (a and c) and a Ppy/COOH-MSN/NGF (b and d) film. The C 1s spectra of Ppy/COOH surfaces indicated the presence of C—C and C—O species in addition to the appearance of minor shouldering of additional species, O—C═O, corresponding to the carboxylic acid functionalities, as shown in FIG. 3(a). The C is spectra after NGF immobilization could be deconvoluted and assigned to contain —C—C species at 285.3 eV, amide-C species at 286.2 eV, and carboxylic acid (O—C═O) species at 287.9 eV; FIG. 3 (b). The presence of amides confirms the adsorption of NGF within the film. Moreover, the analysis through the N is spectra further corroborated the modification of Ppy films. The examination of the high-resolution N 1s core level spectra of Ppy/COOH surfaces confirmed two distinct peaks at 399.0 eV and 400.7 eV corresponding to N—H and C—N, respectively as shown in FIG. 3 (c). Subsequently, Ppy/COOH-MSN/NGF surfaces revealed the existence of NH₂ and amide-N associated with the encapsulation of NGF, FIG. 3 (d). These data indicated that Ppy films with carboxylic acid groups had been successfully prepared in the presence of MSN/NGF. On the other hand, we have presented the construction of macroporous Ppy films using colloidal particles as a template. Such three-dimensional porous Ppy surfaces provided several advantages; i. the pores left by silica particles enhanced surface-to-volume ratios, and ii. carboxylic acid moieties introduced by copolymerization with carboxylic acid-terminated pyrrole monomers could contribute to obtaining higher capacities of reactive functional groups of a hydrophilic nature. Since surface hydrophilicity plays a major role in mediating the cellular adhesion, development, and signaling, surface modification using hydrophilic ligands would promote the cellular response to biomaterials. Furthermore, this could induce the direct coupling of drugs or biomolecules to those surfaces as a beneficial bridge. The performance of macroporous Ppy films can be further extended by covalently conjugating them with nerve growth factor (NGF). After coating particle templates with polypyrrole layers, the treatment with an HF aqueous solution allowed the template to be dissolved, leaving voids in the film in a randomly dispersed way as shown in FIG. 4 (a). The failure to produce ordered channels connecting the pores might have been due to either the aggregation of the adsorbed particles (which would be induced during electrochemical deposition) or incomplete removal of the template. We further explored the electrochemical behaviors of porous Ppy/COOH films using cyclic voltammetry (CV) to determine the ability of surface-modified Ppy films to transfer electrons through the chemical reactivity of the electroactive species.

Turning now to FIGS. 4(a)-4(f), the CV of thin film polymers was defined by irreversible oxidation and reduction waves, where positions and redox peak currents were strongly influenced by the nature of the electrolyte solution. FIG. 4(b) shows the change of CV of differently modified Ppy films conducted in ferricyanide probe solutions as an indicator. Bare ITO surfaces exhibited the obvious magnitude of the oxidation and reduction current. After the self-assembly of MSNs onto ITO surfaces, and subsequent electrodeposition of Ppy (Ppy/COOH-MSN), the redox current magnitude decreases dramatically, which was quite consistent with our previous study. This was due to the response of the ferricyanide probe solution relative to the electrode surface. The template composed of silica nanoparticles might act as a barrier between the ferricyanide species in solution and the ITO electrode consequently inhibiting efficient electron transfer. After removing the silica nanoparticles and obtaining porous Ppy/COOH, the normal redox current of ferricyanide was achieved, indicating the electrons of Fe(CN)₆^{4-/3-} reached the ITO surface through its porosity. On the other hand, NGF-immobilized porous Ppy/COOH showed a slight decrease in the current magnitude due to the steric effects of NGF on the surface. Such porous Ppy films have been analyzed using XPS to understand the composition of the surface, FIG. 4(c-f). Similarly with the results observed in FIG. 3, NGF immobilization on porous films via carbodiimide chemistry showed obvious difference in both C 1s and N 1s spectra by the appearance of amide (—N—C═O—) groups that could arise from chemical conjugation with NGF. Table 1 summarizes and compares various physical properties of modified Ppy films including electrical conductivity and water contact angle.

TABLE 1
The comparison of electrical conductance and water
contact angle of various types of Ppy/COOH films.

| | Conductance | Water contact |
|---|---|---|
| | (S/cm) | angle (° C.) |
| Ppy/COOH | 5.81 +/− 1.04 | 54 +/− 1.7 |
| Porous Ppy/COOH | 3.14 +/− 0.52 | 64 +/− 3.8 |
| Porous Ppy/COOH-NGF | 3.06 +/− 0.08 | 52 +/− 1.4 |
| Porous Ppy/COOH-MSN | 2.57 +/− 0.36 | 51 +/− 3.3 |
| Porous Ppy/COOH_MSN/NGF | 2.83 +/− 0.29 | 55 +/− 2.5 |

The conductivity of Ppy/COOH films was 5.81 S/cm, which is slightly lower than that of Ppy film (7.17 S/cm), partly due to the fact that the presence of the carboxylic acid group at the α-position is likely to induce the conformational alteration and consequently lead to disruption of π-conjugation during polymerization. Meanwhile, electrical conductance of porous Ppy/COOH films corresponded to the 3.14 S/cm, where high porosity was responsible for the decrease in electrical transport in the film. Similarly, the conductivity of Ppy films deposited on the arrays of MSN-NGF decreased with increasing insulating behavior associated with the assembly of MSNs. The water contact angle was examined to define the hydrophilicity after each modification. In comparison with the hydrophobicity of Ppy film (a water contact angle of 76°), co-deposition with carboxylic acidic-Py molecules resulted in a decrease below 64°, indicating the exposure of hydrophilic moieties and carboxylic acids to the surface. On the other hand, the coupling with NGF likely enhanced the hydrophilic nature of Ppy films.

### Effect of Cell Adhesion and Neurite Outgrowth on Ppy/COOH Surfaces

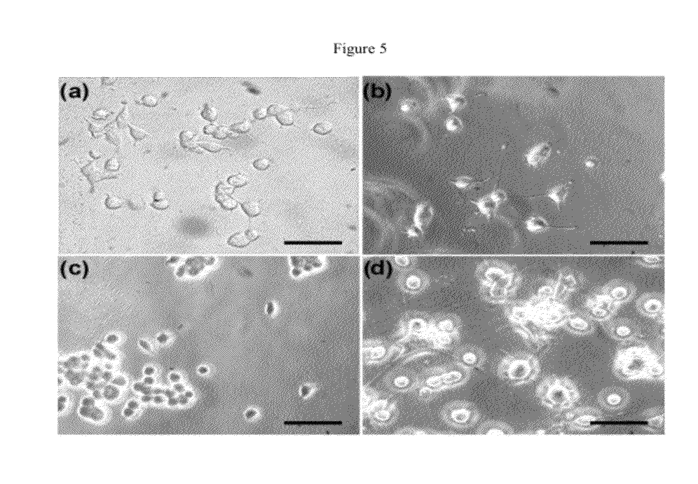

The attachment, growth, and extension of neurites from PC 12 cells on surface-modified Ppy films are shown in FIG. 5. FIG. 5 compares the morphology of a population of PC 12 cells on porous Ppy/COOH immobilized with 1.28 ng/mm² of NGF (Ppy/COOH-NGF) and Ppy/COOH-MSN/NGF films after 2 days of seeding. Surface fuctionalization with NGF facilitated progressive cell growth and development resulting in pronounced differentiation of PC 12 cells. This occurred regardless of the exposure of their substrates to electrical stimulation as shown in FIG. 5A-B. In addition, the uneven surface morphology of porous films (less than 1 μm), as well as the presence of carboxylic acid functionalities, would produced positive cell responsed on that surface. FIG. 5 (c-d) shows electrical field-dependent behavior of PC 12 cells grown on MSN/NGF-encapsulated Ppy/COOH films with distinguishable differences in the morphology and shape.

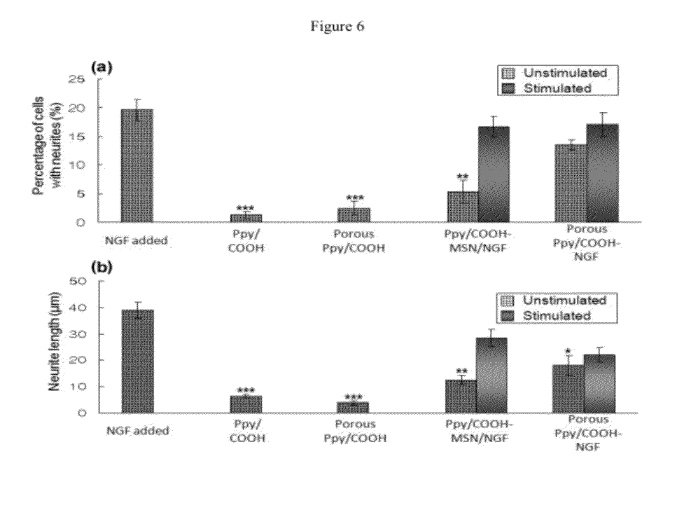

Turning now to FIG. 5, Optical micrographs of PC 12 cell cultured on (a) and (b) porous Ppy/COOH films surface conjugated with NGF and (c) and (d) Ppy/COOH-MSN/NGF films in the absence (a and c) or presence of electrical stimulation (b and d). Scale bar is 50 μm. Notably, when compared to those grown without electrical potential, the application of the external field facilitated cell spreading and neurite extension, demonstrating the morphology expected for PC 12 cells grown with NGF in the medium. Along with the initial evaluation of morphological difference, qualitative studies of neurite outgrowth were performed, as shown in FIG. 6. As a control, applicants used cell populations exposed to NGF for the entire duration of the experiment. Interestingly, we did not find a significant difference in: 1) the number of cells with neurites, and 2) the lengths of their neurites when comparing cell populations grown on porous Ppy/COOH-NGF and Ppy/COOH-MSN/NGF in the presence of electrical stimulation.

Turning now to FIG. 6, Neurite extension from PC 12 cells grown on various types of Ppy/COOH films in the absence or presence of electrical stimulation after 7 days in culture. (a) Percentage of cells with neurites. (b) Neurite length of PC 12 cells grown on several different surfaces. ***p<0.001, **p<0.01, *p<0.05. This result confirmed that PC 12 cells cultured on either NGF-immobilized porous Ppy/COOH surfaces or MSN/NGF-encapsulated Ppy/COOH surfaces established a proliferative state typical of the normal differentiation of these cells. Said another way, NGF-conjugated porous Ppy/COOH-NGF film provided the necessary cytokine to support typical neurite extension, growth cone differentiation, and branching as when cells are cultured in normal media containing NGF. However, in the case of Ppy/COOH-MSN/NGF surfaces, electrical stimulation was necessary to achieve such a permissive substrate by electro-induced diffusion of NGF. Cellular morphology when nerve cells were grown on Ppy/COOH-NGF was not changed as significantly as that of Ppy/COOH-MSN/NGF in response to an electrical potential. Bare Ppy/COOH and porous Ppy/COOH in the absence of NGF did not produce neurite outgrowth. Based on these results, we are preparing carboxylic acid-terminated porous Ppy films with different porosity and pore diameter to behave as a bioactive scaffold that can regulates the survival and differentiation of cells utilizing the additional benefit of electrical stimulation.

Applicants have confirmed the bioactivity of NGF released from Ppy/MSN-NGF composites. In particular, it was apparent that a colloid-based array was associated with the variation in cell adhesion and proliferation, even in the absence of NGF. But when NGF was encapsulated within the composite, direct delivery of NGF to the local area was accomplished and cell growth with enhanced neurite sprouting was encouraged. In this application, NGF-absorbed surfaces combined with a subthreshold electrical potential significantly released this growth factor in response to electrical stimulation. The extensive influence on neural growth by applying steady DC electric fields has been investigated and directed at improving functional recovery in the nervous system. Applicants use an electrical potential to trigger higher levels of NGF release into the extracellular space. In order to examine the effectiveness of electrical stimulation, a solution of NGF (50 ng ml-1) was added to the medium of control Ppy surface and adhered cells were observed 24 h after the treatment. A statistically significant difference between the values of neurite extensions of cell grown on ‘as-prepared’ Ppy surfaces was not achieved.

However, cells grown on electrically stimulated Ppy/MSN-NGF composite showed a statistically significant increase in the amount of neurite extension and growth compared to nerve cells cultured without electrical stimulation. This suggested that subsequent electrical stimulation resulted in elevated release levels of NGF. The effect of electrical stimulation induces an approximately 40% increase in the cell population possessing neurite extensions. The contraction and expansion process of Ppy in response to electrical stimulation enhanced the release of NGF from individual MSN-NGF coated with Ppy. The controlled-release profile of NGF from Ppy/MSN-NGF composites was examined with consideration of the electrical stimulation. NGF release behavior showed linear enhancement as the concentration of MSN-NGF embedded in Ppy film increased.

It will be appreciated that the fabrication of carboxylic acid-functionalized Ppy films using the self-assembly of silica nanoparticles as a template results in electrosensitive delivery devices for NGF, as well as other potential biomedically active chemicals. The incorporation and optional removal of silica particles enables users to selectively engineer the surface properties of the Ppy film. Co-polymerization with carboxylic acid moieties allow resultant two dimensional and/or three dimensional films to possess hydrophilicity which is of importance for various biological applications. Meanwhile, porous Ppy/COOH films further modified with NGF proved that an interactive substrate can also be achieved for the growth and differentiation (perhaps even selected mortality) of cells. Such specially fabricated and electrically stimulated Ppy/COOH films are a significant advancement in the use of biologically active molecules in a medical/biological context.

What is claimed is:

1. A porous polypyrrole composition operable to selectively deliver a selected molecule, comprising:
a. a carboxylic acid-terminated polypyrrole composition comprising a carboxylic acid-functionalized polypyrrole electrochemically deposited upon a mesoporous silica nanosphere array, the carboxylic acid-terminated polypyrrole operational to change conformation upon application of an electrical charge.

2. The porous polypyrrole composition of claim 1, wherein the mesoporous silica nanosphere array is charged with at least one dopant ion.

3. A carboxylic acid-terminated polypyrrole delivery system produced through the process of:
a. producing ordered arrays of mesoporous silica nanoparticles as a template; and
b. electrochemically polymerizing polypyrrole onto the mesoporous silica nanoparticle template.

4. The carboxylic acid-terminated polypyrrole delivery system of claim 3, wherein the step of electrochemically polymerizing polypyrrole onto the mesoporous silica nanoparticle template is performed in an aqueous solution comprising at least one dopant ion.

5. The carboxylic acid-terminated polypyrrole delivery system of claim 4, wherein the dopant ion is one or more neural growth factors.

6. The carboxylic acid-terminated polypyrrole delivery system of claim 4, wherein the electrochemically polymerized polypyrrole is removed from the mesoporous silica nanoparticle template by dipping the electrochemically polymerized polypyrrole in an acidic treatment.

7. The carboxylic acid-terminated polypyrrole delivery system of claim 6, wherein the polymerized polypyrrole is deposited on a surgically implantable object.

8. The carboxylic acid-terminated polypyrrole delivery system of claim 7, wherein the surgically implantable object is inserted into a patient to deliver the at least one dopant ion to a patient.

9. The carboxylic acid-terminated polypyrrole delivery system of claim 8, wherein the dopant ion is released from the polymerized polypyrrole upon electrical stimulation of the implantable object.

10. The carboxylic acid-terminated polypyrrole delivery system of claim 9, wherein the polypyrrole is electrochemically polymerized polypyrrole onto the mesoporous silica nanoparticle template in the presence of a carboxylic acid.
